(19) United States
(12) Patent Application Publication
(10) Pub. No.: US 2026/0256667 A1
(43) Pub. Date: Sep. 3, 2026

(54) ORAL CARE COMPOSITION FOR INCREASING SELF CONFIDENCE

(71) Applicant: GIVAUDAN SA, VERNIER (CH)

(72) Inventors: Stephen David WATKINS, Ashford (GB); Lucy AKINYI, Ashford (GB)

(21) Appl. No.: 19/164,671

(22) PCT Filed: Mar. 14, 2024
(86) PCT No.: PCT/EP2024/056834

§ 371 Date: Sep. 12, 2025

(30) Foreign Application Priority Data
Mar. 15, 2023 (GB) 2303792.2

(51) Int. Cl. A61K 8/42; A61Q 11/00

(52) U.S. Cl. CPC A61K 8/42; A61Q 11/00

(57) ABSTRACT

The present invention relates to use of an oral care composition comprising (1R,2S,5R)—N-(4-cyanomethylphenyl)-p-menthane carboxamide to increase the level and/or duration of an individual's self-confidence.

The present invention relates to the use of an oral care composition comprising (1R,2S,5R)—N-(4-cyanomethylphenyl)-p-menthane carboxamide. In particular, the invention relates to the use of an oral care composition comprising (1R,2S,5R)—N-(4-cyanomethylphenyl)-p-menthane carboxamide to increase an individual's self-confidence.

### BACKGROUND OF THE INVENTION

Breath odour varies widely throughout the daily circadian rhythm and when it is beyond the level of social acceptance, it is termed bad breath or oral malodour. People with better oral health appear to have better experiences with human interaction, and consequently, higher self-esteem and self-confidence. In consumer marketing, positioning of oral care brands with social self-confidence related pack claims have been mainly linked to the product's ability to enhance self-confidence through delivery on core category benefits targeting for example mouth feeling, smelling and tasting fresh (to self and others); having clean, healthy mouth and attractive whiter teeth, the pack claims mainly being a demonstration of confident behaviour related to social setting versus a feeling.

Self-confidence is considered one of the most influential motivations and regulators of behaviour in people's everyday life. Self-confidence is the ‘belief that one can successfully execute a specific activity, rather than a global trait that accounts for overall performance optimism’.

It is known that just the thought of a bad breath problem existing is enough to influence an individual's self-confidence (Mckeown L. (2003) Social relations and breath odour).

Teeth brushing alone has been shown to be enough to improve self-confidence (Taylor, P. et al., The Journal of Social Psychology, 2021, 182-196). However, most studies have evaluated only the immediate effects of teeth brushing on self-confidence (i.e., immediately after teeth brushing). These studies also did not directly measure self-confidence, relying only on correlations with the desired percept.

There is a need to directly correlate self-confidence with the actual consumer product. In recent years, methodologies have been developed to measure individual's self-confidence, such as a “self-report self-confidence measure”, which is administered in questionnaire format and the respondent is asked to report their levels of confidence. Self-report confidence measures capture an overarching self-assessment of confidence, requiring reflection upon personal experiences and tendencies. Typically, items on self-report self-confidence scales ask respondents to consider their confidence in particular tasks or areas and select the extent to which they agree with statements or questions in relation to their own behavior. Such a methodology is, therefore, particularly well-suited for measuring an individual's self-confidence.

Cooling agents are largely tasteless and odourless, nevertheless they have been used to create a cooling effect of a consumer products such as a toothpaste or a mouthwash. Their use in consumer products can also influence the affective state of consumers, influencing for example moods and emotions and evoking positive memories and feelings of well-being.

It is an objective of the present invention to use cooling agents, in particular (1R,2S,5R)—N-(4-cyanomethylphenyl)-p-menthane carboxamide, in an oral care composition in order to increase the level of self-confidence as well as to increase the longevity of the self-confidence of an individual.

### SUMMARY OF THE INVENTION

In one aspect, use of an oral care composition comprising (1R,2S,5R)—N-(4-cyanomethylphenyl)-p-menthane carboxamide to increase the level of an individual's self-confidence is provided.

In another aspect, use of an oral care composition comprising (1R,2S,5R)—N-(4-cyanomethylphenyl)-p-menthane carboxamide to increase the duration of an individual's self-confidence is provided.

In yet another aspect, use of an oral care composition comprising (1R,2S,5R)—N-(4-cyanomethylphenyl)-p-menthane carboxamide to increase both the level and the duration of an individual's self-confidence is provided.

### BRIEF DESCRIPTION OF THE DRAWINGS

FIG. 1 shows a comparison between the level of breath freshness measured at different times over 60 minutes after teeth brushing with flavoured tooth paste alone and with flavoured tooth-paste comprising (1R,2S,5R)—N-(4-cyanomethylphenyl)-p-menthane carboxamide at 150 ppm and 250 ppm.

FIG. 2 shows a comparison between the level of self-confidence measured at different times over 60 minutes after teeth brushing with flavoured tooth paste alone and with flavoured tooth-paste comprising (1R,2S,5R)—N-(4-cyanomethylphenyl)-p-menthane carboxamide at 150 ppm and 250 ppm.

### DEFINITIONS

“Self-confidence” as used herein relates to the firmness or strength of belief about one's competence (‘competence’; ‘ability to do something successfully or efficiently’).

The “level” of self-confidence relates to the intensity of the belief about one's competence.

The “duration” of self-confidence relates to the length of time one believes they feel any measurable level of self-confidence.

All percentages and ratios are calculated by weight unless otherwise indicated.

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

The applicant has surprisingly and unexpectedly found that employing (1R,2S,5R)—N-(4-cyanomethylphenyl)-p-menthane carboxamide in an oral care composition leads to an increase in the level, the duration or both the level and duration of an individual's self-confidence.

Therefore, it is desirable to provide oral care compositions comprising (1R,2S,5R)—N-(4-cyanomethylphenyl)-p-menthane carboxamide to increase the level of an individual's self-confidence, as well as to increase the period of time in which the individual feels self-confident.

### Cooling Agents

Cooling compounds, that is, chemical compounds that impart a cooling sensation to the skin or the mucous membranes of the body, are well known in the art and are widely used in a variety of products such as foodstuffs, tobacco products, beverages, dentifrices, mouthwashes and toiletries.

Examples of such agents have been disclosed, for example, in Leffingwell, J. C., Perfumer & Flavorist, 2004, 39, 34-43 and in the Research Disclosure RD 691054, the disclosures of which are herein incorporated by reference.

Commercially well-known coolants are selected from N-substituted p-menthane carboxamides. Particularly important compounds include N-(4-cyanomethylphenyl) p-menthanecarboxamide, N-(4-sulfamoylphenyl) p-menthane-carboxamide, N-(4-cyanophenyl) p-menthanecarboxamide, N-(4-acetylphenyl) p-menthanecarboxamide, N-(4-hydroxymethylphenyl) p-menthanecarboxamide, N-(3-hydroxy-4-methoxyphenyl) p-menthanecarboxamide, or mixtures thereof. N-(4-cyanomethylphenyl) p-menthanecarboxamide is commercially available from Givaudan under the trade name EverCool™ 180. Other important N-substituted p-menthane carboxamides include menthone glycerol acetal (for example, sold as Frescolat® MGA by Haarmann & Reimer), N-(4-cyanomethylphenyl)-p-menthanecarboxamide or N-(4-cyanomethylphenyl)-5-methyl-2-(1-methylethyl)cyclohexanecarboxamide (for example, commercially available from Givaudan), N-(2-(pyridin-2-yl)ethyl-3-p-menthanecarboxamide (for example, commercially available from Givaudan), N-(4-sulfamoylphenyl)-p-menthanecarboxamide, N-(4-cyanophenyl)-p-menthanecarboxamide, N-(4-acetylphenyl)-p-menthanecarboxamide, N-(4-hydroxymethylphenyl)-p-menthanecarboxamide, N-(3-hydroxy-4-methoxyphenyl)-p-menthanecarboxamide, 2-Isopropyl-N,2,3-trimethylbutyramide (for example, known as WS-23); N-Ethyl-p-menthane-3-carboxamide (for example, known as WS-3); Ethyl 3-(p-menthane-3-carboxamido)acetate (for example, known as WS-5), menthyl lactate (for example, commercially available as Frescolat® ML by Haarmann & Reimer), Menthoxypropane-1,2-diol (for example, commercially available as Coolant Agent 10 by Takasago International), p-Menthane-3,8-diol (for example, commercially available as PMD38)—Takasago International, Isopulegol (for example, commercially available under the name “Coolact P®” by Takasago International), (1R,2S,5R)-2-isopropyl-5-methyl-N-(2-(pyridyn-2-yl)ethyl)cyclohexane-1-carboxamide (commercially available from Givaudan under the trade name EVERCOOL 190), (1-glyceryl-p-mentane-3-carboxylate), (ethyleneglycol-p-methane-3-carboxylate), (N-t-butyl-p-menthane-3-carboxamide), (N-(4-,ethoxyphenyl)-p-menthane-3-carboxamide), 3-(1-menthoxy) propane-1,2-diol, 3-(1-Menthoxy)-2-methylpropane-1,2-diol, menthyl pyrrolidone carboxylate) (for example, commercially available as Questice®), (1R,3R,4S)-3-menthyl-3,6-dioxaheptanoate (for example, commercially available from Firmenich), (1R,2S,5R)-3-menthyl methoxyacetate (for example, commercially available from Firmenich), (1R,2S,5R)-3-menthyl 3,6,9-trioxadecanoate (for example, commercially available from Firmenich), (1R,2S,5R)-menthyl 11-hydroxy-3,6,9-trioxaundecanoate (for example, commercially available from Firmenich), (1R,2S,5R)-3-menthyl(2-hydroxyethoxy)acetate (for example, commercially available from Firmenich), Cubebol (for example, commercially available from Firmenich), 1-[2-hydroxyphenyl]-4-[2-nitrophenyl-]-1,2,3,6-tetrahydropyrimidine-2-one), 4-methyl-3-(1-pyrrolidinyl)-2 [5H]-furanone (for example, known as Icilin or AG-3-5), menthyl lactate, menthone glycer—in acetal, L-Monomenthyl succinate, L-monomenthyl glutarate, 3-1-menthoxypropane-1,2-diol (for example, known as Coolact 10), 2-1-menthoxyethanol (for example, known as Cooltact 5), and mixtures thereof. Additional non-menthol coolants are described in U.S. Pat. No. 7,414,152, US20100086498 A1 and WO2010/128026 A2, which coolants are incorporated herein by reference. Other cooling agents are disclosed in U.S. Pat. Nos. 4,136,163 and 4,150,052 and 4,178,459 and 4,190,643 and 4,193,936 and 4,226,988, 4,230,688 and 4,032,661 and 4,153,679 and 4,296,255, 4,459,425, 5,009,893, 5,266,592, 5,698,181, 5,725,865 and 5,843,466, 6,231,900, 6,277,385, 6,280,762 and 6,306,429 and 6,432,441, 6,455,080 and 6,627,233 and 7,078,066, 6,783,783, 6,884,906, 7,030,273, and 7,090,832; US 2004/0175489, US 2004/0191402, US 2005/0019445, US 2005/0222256 and US 2005/0265930, US 2006/0159819, and US 2006/0249167; EP 1689256 A1; and. WO 2005/082154 A1 o, WO 2005/099473 A1, WO 2006/058600 A1, WO 2006/092076 A2, and WO 2006/125334 A1, all of which being incorporated herein by reference.

In particular, (1R,2S,5R)—N-(4-cyanomethylphenyl)-p-menthane carboxamide (also referred to as EC 180) is disclosed in WO 2005/049553 as exhibiting a strong and long-lasting cooling effect, especially when employed on oral care compositions such as mouthwashes or toothpastes. (1R,2S,5R)—N-(4-cyanomethylphenyl)-p-menthane carboxamide provides a long-lasting cooling sensation in the mouth but has no aroma (flavour) of its own and so does not contribute to pleasant-smelling breath. Surprisingly, it has now been found that, despite having no aroma, (1R,2S,5R)—N-(4-cyanomethylphenyl)-p-menthane carboxamide enhances self-confidence.

Having fresh smelling breath often contributes to a large extent to an individual's self confidence in social interactions and situations. (1R,2S,5R)—N-(4-cyanomethylphenyl)-p-menthane carboxamide contributes to none of the benefits associated with self-confidence such as clean, healthy mouth and pleasant-smelling breath, however it has been found that (1R,2S,5R)—N-(4-cyanomethylphenyl)-p-menthane carboxamide enhances self-confidence when compared to flavour alone and that this enhanced self-confidence also lasts for an extended period of time.

The present invention shows that employing (1R,2S,5R)—N-(4-cyanomethylphenyl)-p-menthane carboxamide in oral care compositions leads, in addition to the known cooling effect, to an increase in the self-confidence of the consumer. The increased effect is related to both the magnitude as well as to the duration of the effect.

### Flavour Ingredient

Flavour ingredients are incorporated in oral care compositions, e. g. toothpaste, mouthwash and chewing gum, amongst others, to impart a signal to a consumer that the product may deliver freshness and cleanliness upon use. If a product has a fresh taste and smell, then these are key indicators to consumers that their teeth, mouth and breath will be clean with use.

Examples of flavour ingredients include natural flavors, artificial flavors, spices, seasonings, and the like. Exemplary flavor ingredients include synthetic flavor oils and flavoring aromatics and/or oils, oleoresins, essences, and distillates, and a combination comprising at least one of the foregoing.

Flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents include artificial, natural and synthetic fruit flavors such as vanilla, and citrus oils including lemon, orange, lime, grapefruit, yuzu, sudachi, and fruit essences including apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya, and so forth.

Additional exemplary flavors imparted by a flavoring composition include a milk flavor, a butter flavor, a cheese flavor, a cream flavor, and a yogurt flavor; a vanilla flavor; tea or coffee flavors, such as a green tea flavor, an oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; a nut flavor such as an almond flavor, a hazelnut flavor, a macadamia nut flavor, a peanut flavor, a pecan flavor, a pistachio flavor, and a walnut flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor.

Generally any flavoring or food additive (including food colors) such as those described in “Essential guide to food additives”, Third edition, 2008, page 101-321 (ISBN: 978-1-905224-50-0) by Leatherhead Food International Ltd., can be used. The publication is incorporated herein by reference.

Many flavour types are known and may be exploited in the oral care area, but flavour compositions which include mint oils, e. g. peppermint and spearmint oils, are particularly popular in oral care compositions. Mint oils are typically used to impart a connotation of freshness as they naturally contain menthol which has physiological coolant properties. Often this impression of freshness is enhanced by the inclusion of additional menthol, regardless of the flavour type.

A typical peppermint flavor may contain anethole (up to 20%, e.g. 5 to 15%), menthol (up to 70%, (e.g., 20-50%), Mentha arvensis (up to 70%, e.g., 10-30%), Mentha piperita (up to 50%, e.g. 10-30%), Mentha spicata and/or L-carvone (up to 20%, e.g. 5-15%), and other flavor or fragrance ingredients.

A typical spearmint flavor may contain anethole (up to 20%, e.g., 5 to 15%), menthol (up to 50%, e.g., 15-30%), Mentha spicata and/or L-carvone up to 50%, e.g. 20-40%), Mentha arvensis and/or Mentha piperita (up to 50%, e.g. 10-30%), and other flavor or fragrance ingredients.

In one embodiment, the flavor ingredient is present in an oral care composition in the range from about 0.02% by weight to about 10% by weight, preferably in the range from about 0.2% by weight to about 5.0% by weight and more preferably from about 0.3% by weight to about 3.0% by weight.

The flavour is particularly important in providing a pleasant-smelling breath but WO 2004/073669A1 shows that the oral residency time for flavour is relatively short, often less than ten minutes, so the self-confidence correlated with the breath freshness provided by flavored tooth-paste-only diminishes rapidly after use.

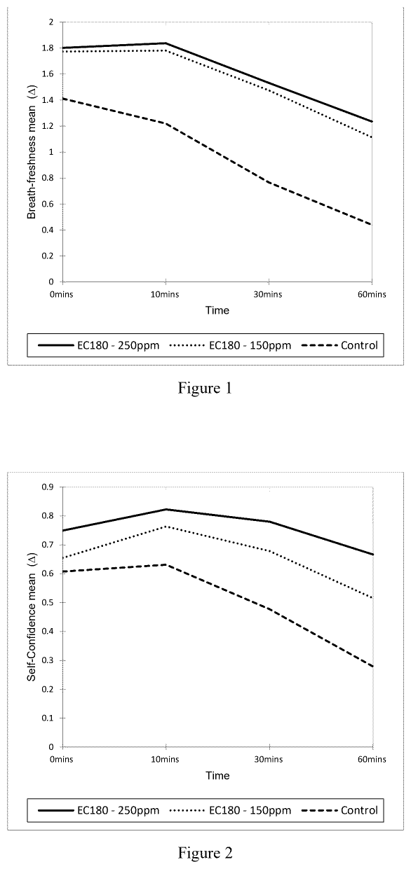

This has been confirmed by monitoring both the level of breath freshness and the level of self-confidence reported by a number of participants in the present study as shown by the dashed line in FIGS. 1 and 2, which demonstrate that despite a drop in the level of breath freshness, the level of self-confidence slightly increased 10 min after teeth brushing. Thereafter, the levels of both breath freshness and self-confidence decreased significantly over a period of 60 minutes.

Therefore, the present invention shows that teeth brushing with flavoured tooth paste alone was only enough to maintain a certain level of self-confidence for a short period of time, such as about 10 minutes. However, the level of self-confidence started declining thereafter, the value significantly dropping after about 60 min (FIG. 1).

In contrast, the presence of (1R,2S,5R)—N-(4-cyanomethylphenyl)-p-menthane in a flavoured tooth-paste used for teeth-brushing significantly increased the level of self-confidence 10 minutes after brushing, despite providing the same level of breath freshness over the same period of time (the solid and dotted lines in FIGS. 1 and 2). The level of self-confidence remained elevated for a period of time of up to about 30 min after teeth brushing, an effect still being observed 60 min after teeth brushing, despite the levels of breath freshness steadily declining during the same period of time.

In one embodiment, (1R,2S,5R)—N-(4-cyanomethylphenyl)-p-menthane carboxamide is used at a level of 2 to 800 ppm, optionally 20 to 500 ppm, particularly 50 to 400 ppm, more particularly 100 to 300 ppm, even more particularly 150 to 250 ppm, based on the weight of the oral care composition.

### Oral Care Composition

The oral care composition, such as a toothpaste or a mouthwash may be formulated in a conventional manner as is well known to those skilled in the art. In one embodiment, the oral care composition comprises, in addition to (1R,2S,5R)—N-(4-cyanomethylphenyl)-p-menthane carboxamide, a flavour ingredient.

In one embodiment, the oral care composition further comprises at least one orally acceptable carrier. Orally acceptable carriers may comprise one or more compatible solid or liquid excipients or diluents which are suitable for topical oral administration. By “compatible,” as used herein, is meant that the components of the composition are capable of being commingled without interaction in a manner which would substantially reduce stability and/or efficacy. The carriers can include the usual and conventional components of dentifrices, non-abrasive gels, sub-gingival gels, mouthwashes or rinses, mouth sprays, chewing gums, lozenges and breath mints. The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, etc. as disclosed in e.g., U.S. Pat. No. 3,988,433. Carrier materials for biphasic dentifrice formulations are disclosed in U.S. Pat. Nos. 5,213,790; 5,145,666 and 5,281,410 and in U.S. Pat. Nos. 4,849,213 and 4,528,180. Mouthwash, rinse or mouth spray carrier materials typically include water, flavoring and sweetening agents, etc., as disclosed in, e.g., U.S. Pat. No. 3,988,433. Lozenge carrier materials typically include a candy base; chewing gum carrier materials include a gum base, flavoring and sweetening agents, as in, e.g., U.S. Pat. No. 4,083,955. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g. U.S. Pat. Nos. 5,198,220 and 5,242,910. Carriers suitable for the preparation of compositions of the present disclosure are well known in the art. Their selection will depend on secondary considerations like taste, cost, and shelf stability, and the like. Further suitable types of orally acceptable carrier materials or excipients are listed in WO2010/059289, in particular on page 17-31, which is incorporated by reference.

In one embodiment, the oral care composition is a tooth paste.

In one embodiment, the self-confidence is self-confidence in a person's oral health.

The present invention is further illustrated by means of the following non-limiting examples

### Method of Measurement

Individual's thoughts and feelings in relation to their self-confidence as per their perceived oral health (such as breath) were measured using a 9-item subscale of the modified Competitive State Anxiety Questionnaire (CSAI-2).

The Competitive State Anxiety Questionnaire (CSAI) was designed originally to examine anxiety in competitive performance by Martens et al. (Martens, R. B. et al, (1990), Development and validation of the competitive state anxiety inventory-2. Competitive anxiety in sport, Human Kinetics, Chapaign, 117-190). A third sub-scale labelled ‘self-confidence’, which is characterized by positive expectations of success, was then added (CSAI-2) (Cerin, E., J. Appl. Sport psychology, 2003, 15, 223-238). CSAI-2 has become the tool of choice to measure anxiety before an intensive performance (such as sports or musical). A reduced version of the questionnaire, i.e. employing just one subscale, has been validated by Arruza Gabilondo et al. (Arruza Gabilondo, J. A. et al, Revista Internacional de Medicina y Ciencias de la Actividad Fisica y del Deporte, 2012, 47, 539-556). A recent oral care study (Dumitrescu, A. L. et. al, Int. J. Dent. Hygiene, 2012, 22-29) used the 9-item self-confidence subscale from the Competitive State Anxiety Questionnaire-2 (CSAI-2) in examining self-confidence.

The nine items in the Self-confidence scale include five that describe positive expectations (“I feel self-confident”, “I am confident I can meet the challenge”, “I'm confident about performing well”, “I'm confident because I mentally picture myself reaching my goal”, and “I'm confident about coming through under pressure”), and four that describe a generalised feeling of calmness (“I feel calm”, “I feel comfortable”, “I feel secure”, “I feel mentally relaxed”). All items were rated on a 4-point scale anchored by 1 (“Not at all”) and 4 (“Very much so”).

### Participants

This home use test study was conducted in England in July/August 2022. Twenty-six participants were pre-screened to ensure that they had no underlying dental problems.

### Study Design and Procedures

A repeated measures design, with a randomised repeated trial per product was used. Three toothpastes were prepared with:
Control: peppermint flavour
EC180—150 ppm: peppermint flavour+150 ppm EC180 (concentration in toothpaste)
EC180—250 ppm: peppermint flavour+250 ppm EC180 (concentration in toothpaste)

The toothpaste base formula is shown in Table 1.

TABLE 1
Toothpaste Base Formula

| Ingredient | wt % |
|---|---|
| Trisodium Phosphate dodecahydrate | 0.2 |
| Sorbitol | 50.0 |
| Precipitated Silica - Sorbosil ™ AC77 (Surfachem) | 8.0 |
| Precipitated Silica - Sorbosil ™ TC15 (Surfachem) | 9.0 |
| Sodium Carboxy Methyl Cellulose 9M31XF (Ashland) | 0.8 |
| Titanium Dioxide | 1.0 |
| Sodium Lauryl Sulphate | 1.5 |
| PEG 1500 (Kilo) | 5.0 |
| Demin Water | 23.5 |
| Flavour | 1.0 |

The flavor compositions, also including (1R,2S,5R)—N-(4-cyanomethylphenyl)-p-menthane carboxamide (EC180) are shown in Table 2.

TABLE 2
Flavor Compositions Including EC180

| | | EC180 - | EC180 - |
|---|---|---|---|
| | | 150 | 250 |
| | Control | ppm | ppm |
| Ingredient | (wt %) | (wt %) | (wt %) |
| Anethole | 9.00 | 9.00 | 9.00 |
| L-Carvone | 3.00 | 3.00 | 3.00 |
| L-menthol | 25.00 | 25.00 | 25.00 |
| Orange Oil (Brazilian) | 2.80 | 2.80 | 2.80 |
| Spearmint Oil | 6.00 | 6.00 | 6.00 |
| Vanillal | 0.10 | 0.10 | 0.10 |
| Peppermint Chassis | 51.60 | 51.60 | 51.60 |
| Propylene Glycol | 2.50 | 1.00 | 0.00 |
| (1R,2S,5R)-N-(4-cyanomethylphenyl)-p- | 0.00 | 1.50 | 2.50 |
| menthane carboxamide | | | |

Participants were given duplicate tubes of each toothpaste (6 tubes in total). All products were assigned different codes and presented in white tubes to prevent colour as a confounding factor.

The participants did the test each morning in their homes as per their usual teeth cleaning time (between 7:00 am and 10:00 am) and were asked to refrain from using other oral practices apart from brushing with test product. Testing was done with one product a day with a one-day separation left before testing the next product. A questionnaire was sent to them via an online link for them to complete before teeth cleaning and after teeth cleaning. Responses before teeth cleaning were recorded as their baseline scores.

The data for this test was collected through an online questionnaire set up via SurveyMonkey Audience. The questionnaires on breath-freshness and self-confidence was set up for participants to respond to at the five time-points: (i) pre-brush baseline (ii) 0 minutes immediately post-bush (iii) 30 minutes post-brush (iv) 60 minutes post-brush. A total of 47 datasets per product was input for analysis and 5 incompletes were removed.

### Measurements

### Breath-Freshness Assessment

Breath freshness was self-evaluated with a set of three questions asking (i) whole mouth clean feel; (ii) natural freshness in mouth; and (lii) cooling sensation in the mouth. To obtain a breath self-assessment measure, participants were asked to refrain from drinking and eating as well as oral hygiene practices at least 1 hour before teeth brushing and for the whole period of assessment after teeth brushing. Breath assessment recorded before teeth brushing was categorized as the baseline measure.

### Self-Confidence

Self-confidence was measured with the 9-Item subscale of the Modified Competitive State Inventory-2 (CSAI-2). The questionnaire uses a 4-point scale as follows:

1—not at all; 2—somewhat; 3—moderately so; 4—very much so.

### Example 1: Baseline Breath-Freshness Assessment and Self-Confidence Scores

Before brushing (baseline) mean and standard deviation (SD) was calculated across products and tabulated as below in tables 3 and 4. A two-way ANOVA revealed the p-values were above 0.05 indicating no significant differences between products in breath assessment and self-confidence before brushing. This showed that participants' breath-freshness and self-confidence before brushing was rated almost the same across the test days before use of products.

TABLE 3
A two-way ANOVA Baseline (Pre-brush) breath-freshness

| Source of | | | | | P- | |
|---|---|---|---|---|---|---|
| Variation | SS | df | MS | F | value | F crit |
| Between | 0.881009 | 2 | 0.440504 | 1.750681 | 0.177 | 3.061716 |
| Groups | | | | | | |
| Within | 34.7234 | 138 | 0.251619 | | | |
| Groups | | | | | | |
| Total | 35.60441 | 140 | | | | |

TABLE 4
A two-way ANOVA Baseline (Pre-brush) Self-Confidence

| Source of | | | | | P- | |
|---|---|---|---|---|---|---|
| Variation | SS | df | MS | F | value | F crit |
| Between | 0.239734 | 2 | 0.119867 | 0.257409 | 0.773 | 3.061716 |
| Groups | | | | | | |
| Within | 64.26215 | 138 | 0.465668 | | | |
| Groups | | | | | | |
| Total | 64.50188 | 140 | | | | |

Table 5 shows baseline summary statistics (before brushing) of the participants' self-confidence and breath freshness. There were no significant differences in the baseline measures between products in both questionnaires.

TABLE 5
Baseline measure of breath freshness and self-confidence

| Statistic | N | Mean | SD (n) | P-value |
|---|---|---|---|---|
| Self-confidence | | | | 0.773 |
| Control | 47 | 2.312 | 0.731 | |
| EC180 - 150 ppm | 47 | 2.225 | 0.574 | |
| EC180 - 250 ppm | 47 | 2.225 | 0.731 | |
| Breath freshness | | | | 0.177 |
| Control | 47 | 1.504 | 0.550 | |
| EC180 - 150 ppm | 47 | 1.312 | 0.391 | |
| EC180 - 250 ppm | 47 | 1.383 | 0.533 | |

### Example 2: Delta Breath-Freshness Assessment and Self-Confidence Scores

To evaluate a change effect from the baseline (pre-brush) to the different times post-brush, a mean difference between each time-point to the baseline mean score was calculated to obtain delta values.

### Delta Breath-Freshness Assessment

FIG. 1 shows the changes of breath freshness mean delta values of EC180—150 ppm, EC180—250 ppm and Control over time.

A two-way ANOVA (Table 6) revealed that there was a statistically significant difference in mean breath-freshness scores between the toothpaste products (F(2,552)=10.298, p=0.000) and time (F(3,552)=33.322, p=0.000).

Post hoc analysis, Tukey's HSD, a test for multiple comparisons, found that that the mean value of the toothpaste products was significantly different between control product and both levels of EC180 at all time-points after brushing. There were no significant differences between the levels of EC180.

Tukey's Honest Significant Difference (HSD) Test is a post hoc test commonly used to assess the significance of differences between pairs of group means.

TABLE 6
Two-way ANOVA breath freshness assessment

| Source of | | | | | P- | F |
|---|---|---|---|---|---|---|
| Variation | SS | df | MS | F | value | crit |
| Sample | 10.19267 | 2 | 5.096 | 10.298 | 0.000 | 3.012 |
| (Toothpaste | | | | | | |
| products) | | | | | | |
| Columns (Time) | 49.47281 | 3 | 16.491 | 33.322 | 0.000 | 2.621 |
| Interaction | 2.800236 | 6 | 0.467 | 0.943 | 0.464 | 2.115 |
| Within | 273.182 | 552 | 0.495 | | | |
| Total | 335.6478 | 563 | | | | |

### Delta Self-Confidence Assessment

Delta values were calculated as a change from baseline. FIG. 2 shows the changes of self-confidence mean delta values of EC180—150 ppm, EC180—250 ppm and Control.

A two-way ANOVA (Table 7) revealed that there was a statistically significant difference in mean self-confidence scores between the toothpaste products (F(2,552)=37.465, p=0.000) and time (F(3,552)=4.120, p=0.007).

Post hoc analysis, Tukey's HSD, a test for multiple comparisons, found that that the mean value of the toothpaste products was significantly different between control product and EC180 level 250 ppm at time 30 minutes (p=0.071, 90% CI=[−0.576, −0.020]) and at time 60 minutes (p=0.009, 90% CI=[−0.642, −0.119]). There was no statistically significant difference between the toothpaste products at time 0 min and 10 min.

TABLE 7
Two-way ANOVA self-confidence assessment

| Source of | | | | | | |
|---|---|---|---|---|---|---|
| Variation | SS | df | MS | F | P-value | F crit |
| Sample | 29.171 | 2 | 14.586 | 37.465 | 0.000 | 3.012 |
| (Toothpaste | | | | | | |
| products) | | | | | | |
| Columns (Time) | 4.812 | 3 | 1.604 | 4.120 | 0.007 | 2.621 |
| Interaction | 0.765 | 6 | 0.127 | 0.327 | 0.923 | 2.115 |
| Within | 214.903 | 552 | 0.389 | | | |
| Total | 249.651 | 563 | | | | |

It is clear, therefore, that while breath freshness starts decreasing significantly after 10 minutes from brushing with a toothpaste containing EC180 (reaching about 60% of the initial level after 60 minutes from brushing), the person's self-confidence is maintained at an appreciable level (more than about 80%) compared to the level attained immediately after brushing with the toothpaste containing EC180, even after 60 minutes from brushing.

It can also be observed that breath freshness only minimally increases up to 10 minutes after brushing compared to immediately after brushing (0 min), whereas a noticeable increase in the level of self-confidence can be observed 10 minutes after brushing compared to immediately after brushing (0 min), when employing a toothpaste comprising EC180.

1. A method for increasing the level of an individual's self-confidence, wherein the method comprises administering an oral care composition comprising (1R,2S,5R)—N-(4-cyanomethylphenyl)-p-menthane carboxamide to the individual.

2. A method for increasing the duration of an individual's self-confidence, wherein the method comprises administering an oral care composition comprising (1R,2S,5R)—N-(4-cyanomethylphenyl)-p-menthane carboxamide to the individual.

3. A method for increasing the level and duration of an individual's self-confidence, wherein the method comprises administering an oral care composition comprising (1R,2S,5R)—N-(4-cyanomethylphenyl)-p-menthane carboxamide to the individual.

4. The method of claim 1, wherein the individual's self-confidence is self-confidence in their oral health.

5. The method of claim 1, wherein the oral care composition further comprises at least one flavour ingredient.

6. The method of claim 5, wherein the at least one flavour ingredient is selected from the groups consisting of spearmint oil, cinnamon oil, oil of wintergreen, peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; artificial, natural and synthetic fruit flavors, citrus oils, fruit essences, and combinations thereof.

7. The method of claim 1, wherein (1R,2S,5R)—N-(4-cyanomethylphenyl)-p-menthane carboxamide is present at a level of 2 ppm to 800 ppm, based on the weight of the oral care composition.

8. The method of claim 1, wherein the oral care composition is a tooth paste or a mouthwash.

9. The method of claim 8, wherein the oral care composition is a tooth paste.

10. The method of claim 2, wherein the individual's self-confidence is self-confidence in their oral health.

11. The method according to claim 6, wherein the at least one flavour ingredient is selected from the group consisting of vanilla, lemon, orange, lime, grapefruit, yuzu, sudachi apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya and mixtures thereof.

12. The method of claim 7, wherein (1R,2S,5R)—N-(4-cyanomethylphenyl)-p-menthane carboxamide is present at a level of 20 ppm to 500 ppm, based on the weight of the oral care composition.

13. The method of claim 7, wherein (1R,2S,5R)—N-(4-cyanomethylphenyl)-p-menthane carboxamide is present at a level of 50 ppm to 400 ppm, based on the weight of the oral care composition.

14. The method of claim 7, wherein (1R,2S,5R)—N-(4-cyanomethylphenyl)-p-menthane carboxamide is present at a level of 100 ppm to 300 ppm, based on the weight of the oral care composition.

15. The method of claim 7, wherein (1R,2S,5R)—N-(4-cyanomethylphenyl)-p-menthane carboxamide is present at a level of 150 ppm to 250 ppm, based on the weight of the oral care composition.
